(19) United States
(12) Patent Application Publication
(10) Pub. No.: US 2009/0081162 A1
(43) Pub. Date: Mar. 26, 2009

(54) METHOD FOR DOWNREGULATING AN IMMUNE REACTION

(76) Inventor: Michael Tovey, Paris (FR)

Correspondence Address: BROWDY AND NEIMARK, P.L.L.C.;624 NINTH STREET, NW, SUITE 300, WASHINGTON, DC, 20001-5303, US

(21) Appl. No.: 11/661,421

(22) PCT Filed: Aug. 31, 2005
(86) PCT No.: PCT/US2005/031004

§ 371 Date: Dec. 1, 2008

Related U.S. Application Data
(60) Provisional application No. 60/605,513, filed on Aug. 31, 2004.

(51) Int. Cl. A61K 38/21; A61P 31/12; A61P 37/00; A61P 29/00

(52) U.S. Cl. 424/85.6; 424/85.4; 424/85.7

(57) ABSTRACT

Ongoing pathogenic immune reactions and the severity of an incipient or prospective immune reaction may be downregulated or reduced by locally administering Type I interferon at a site remote from the ongoing, incipient or prospective immune reaction. The amount of interferon to be administered is that which is effective to induce trafficking of antigen-presenting cells to the site of interferon administration, and away from the site of the immune reaction. It is administered in the course of the ongoing immune reaction, or concurrently with the provocation of an immune reaction.

### FIELD OF THE INVENTION

The present invention is directed to methods for downregulating an ongoing pathogenic immune reaction, or reducing the severity of an incipient or prospective immune reaction in a patient in need thereof. More particularly, the present invention is related to the accomplishment of such a method by locally administering Type I interferon at a site remote from the site of the ongoing, incipient or prospective immune reaction.

### BACKGROUND OF THE INVENTION

The innate immune system recognizes pathogen associated molecular patterns (PAMPs), complex molecules comprising lipids, carbohydrates and peptides, conserved on microbial pathogens and absent from the host. Recognition of PAMPs occurs through a family of pattern recognition Toll-like receptors (TLR) present on the surface of antigen-presenting cells (APC) including dendritic cells (DC), macrophages, and B-cells (Medzhitov, 2001). Toll-like receptors elicit specific signaling cascades that regulate innate immunity and ultimately result in stimulation and regulation of both T-cell and B-cell responses and the establishment of the adaptive cellular and humoral immune response.

Lipopolysaccharide (LPS:endotoxin), the main component of the outer membrane of Gram-negative bacteria, activates immunity through TLR4 in co-operation with the co-receptor MD2. Viral double-stranded RNA (dsRNA) or synthetic dsRNA molecules, such as poly (I:C), signal through TLR 3. Recognition of bacterial DNA by TLR9 is based on the presence of un-methylated CpG dinucleotides in a particular sequence context. Synthetic oligodeoxynucleotides that contain CpG motifs mimic bacterial DNA and induce a coordinated set of immune responses including activation of innate immunity and acquired Th1 biased cellular and humoral immunity (Medzhitov, 2001).

TLRs have a Toll-ILl receptor-resistance (TIR) motif in their cytoplasmic domains and signal through adapter proteins that also have TIR motifs. Three adapter proteins, MyD88, Tirap (or MAL), and Trif (or TICAM-1), have been identified to date as participating in LPS signaling, while dsRNA signaling occurs through TLR3 and Trif.

Dendritic cells (DC) are professional antigen-presenting cells that link the innate and adaptive immune response. Dendritic cells take up and degrade pathogens or soluble antigens and present the degradation products as peptides in the context of MHC class I and class II antigens to T-cells, thereby initiating T-cell dependent immune responses. A subset of dendritic cells has been characterized with a plasmacytoid morphology that produce high levels of Type I interferon (IFN α/β) in response to virus infection or oligodeoxynucleotides containing un-methylated CpG motifs but do not respond to bacterial extracts or LPS (Diebold et al, 2004). IFN producing pDCs play an essential role in activating NK-cells to destroy virus-infected cells. Plasmacytoid DC are able to differentiate into mature DCs that prime naive CD4+ T-cells either towards a T helper type I (Th1) or Th2 response depending upon the activation stimulus. It has been shown recently that pDCs produce high levels of IFN-α in response to infection with wild type influenza virus even though the NS1 viral protein sequesters dsRNA inhibiting induction of IFN-α through TLR3. This mechanism involves degradation of viral particles by endosomal proteases, liberation of single-stranded RNA (ssRNA) and signaling through TLR7 and MyD88 (Diebold et al 2004). Guanosine (G) and uridine (U) rich synthetic ssRNA oligonucleotides derived from HIV-1 stimulate DCs and macrophages to produce IFN-α via TLR 7 signaling in the mouse and TLR8 in man. Sequence homology suggests that TLR 7 and TLR 8 form a sub-family of TLR9 (Heil et al, 2004).

The adaptive immune response is dependent upon induction of co-stimulatory molecules, including CD40, CD80 (B7-1), and CD86 (B7-2), which are expressed by professional antigen presenting cells, including macrophages and dendritic cells. These molecules help to generate a mitogenic response within T-cells that are exposed to antigen in the context of major histocompatibility (MHC) class I or class II proteins (Mayer et al, 2004).

B-cell activation by protein antigens requires binding of the antigen to the B-cell surface immunoglobulin (B-cell receptor) and co-stimulation by antigen-specific T cells through CD40-CD40 ligand interaction and the action of T-cell derived cytokines. Appropriately activated B-cells proliferate and differentiate into plasma cells or to long-lived memory cells. Recent evidence suggests that the activity of B-cells can be regulated in a T-cell independent manner. Indeed, in common with other antigen presenting cells, B-Cells possess TLR 9 receptors, the natural ligands of which are CpG motifs present within microbial DNA. HSV genomic DNA has been reported to engage TLR 9 and induce IFN-α in pDCs (Diebold et al, 2004, Heil et al, 2004).

Presentation of antigen in the absence of the induction of co-stimulatory molecules leads to tolerance or anergy. Thus, autoreactive T-cells that escape negative selection in the thymus (central tolerance) but later encounter an abundance of self-antigen in the periphery may be either anergized or deleted in a process known as peripheral tolerance. High doses of antigen administered orally can also induce lymphocyte anergy and/or deletion. High dose antigen induced deletion occurs through CD95 (FAS)-dependent caspase activation leading to apoptosis. This process can be blocked by the Th1 cytokine interleukin 12 (IL-12). Anergy occurs as a result of T-cell receptor (TCR) ligation with inadequate co-stimulation, either by cognate interactions between CD80 or CD86 on APCs with CD38 on T-cells, or by soluble cytokines such as IL-2. Low-dose tolerance is mediated by active suppression of the immune response by a sub-class of T-cells known as regulatory T-cells. These cells can be divided into three groups: CD4+CD25+ regulatory T-cells (CD4+ T-cells that co-express high levels of the IL-2 receptor a chain; CD25) that mature in the thymus and that are reactive to self-antigen in the periphery; TGF-α secreting Th3 cells that been implicated in oral tolerance, and TR1 cells that are activated by chronic alloantigen stimulation and that require IL-10 for their growth as well as for their ability to suppress inflammation in models of inflammatory bowel disease (Mayer et al, 2004). Although all three classes of regulatory T-cells can be activated in an antigen-specific manner, and whether they produce immuno-suppressive cytokines or act through cell-surface ligands, they are able to suppress the immune response in their immediate surroundings in an antigen-independent manner. This phenomenon is known as by-stander suppression.

IFN-α produced primarily by pDCs as part of the innate immune response to infectious agents is a powerful polyclonal B-cell activator that induces a strong primary humoral immune response characterized by isotype switching (Le Bon et al, 2001) and protection against virus challenge (ibid). IFN-α secreted by pDCs in response to virus infection has been shown to induce B-lymphocytes to differentiate into antibody producing plasma cells and to be necessary for the production of both specific and polyclonal IgGs in response to influenza infection (Jego et al, 2003). IFN-α has also been shown to be an usually powerful adjuvant when ad-mixed with influenza vaccine and the mixture is injected intramuscularly (Proietti et al, 2002).

The present invention is based on the surprising discovery that injection of IFN-α at a site remote from the site of vaccination profoundly inhibits the antibody response to influenza vaccination. This phenomenon most probably reflects, at least in part, IFN-induced migration of antigen-presenting cells.

### SUMMARY OF THE INVENTION

Accordingly, it is an object of the present invention to provide a method for downregulating an ongoing pathogenic immune response.

It is a further object of the present invention is to provide a method for reducing the severity of an incipient or prospective immune reaction.

It is another object of the present invention to provide a method for the treatment of allergic attack, atopic asthma, allergic rhinitis, or anaphylactic shock that results from exposure to an antigen to which the patient is hypersensitive.

It is yet another object of the present invention to provide a method for the treatment of undesirable inflammation.

It is yet another object of the present invention to provide a method for downregulating the undesirable immunogenic response to the administration of a therapeutic protein which is immunogenic, such as a non-human antibody.

It is yet a further object of the present invention to provide a method to downregulate the immune reaction to organ or tissue transplantation.

It is still a further object of the present invention to provide a method for the treatment of Crohn's disease, or other forms of inflammatory bowel disease.

These and other objects of the present invention are accomplished by means of the local administration of Type I interferon to the patient in need thereof, at a site remote from the site of the ongoing, incipient or prospective immune reaction. The administration should be in the course of the ongoing pathogenic immune reaction or concurrently with the provocation of an immune reaction. The interferon is locally administered at a site remote from the site of the immune reaction. The amount of Type I interferon administered should be an amount effective to induce trafficking of antigen-presenting cells to the site of interferon administration and away from the site of the immune reaction. Preferably, the patient is other than one that has multiple sclerosis.

### BRIEF DESCRIPTION OF THE DRAWINGS

FIG. 1 is a graph showing the effect of IFN-α on the anti-influenza antibody response measured 30 days after vaccination. The four sets of three columns represent measurements of IgG2a, IgA, IgG1, and total IgG. In each group of three columns, the left, or white column measures the total amount of anti-influenza antibody, measured 30 days following intramuscular (im) administration of influenza vaccine (15 micrograms of Vaxigrip) and the substantially simultaneous intraperitoneal (ip) administration of 10⁵ IU IFN-α. The center, or gray column represents the anti-influenza antibody response 30 days following the administration of a mixture of 15 micrograms of Vaxigrip and 10⁵ IU IFN-α injected intramuscularly. The third, or black column measures the anti-influenza antibody response 30 days after im administration of 15 micrograms of Vaxigrip alone.

FIG. 2 is a graph showing the fold increase of Crg2 mRNA at various times after simultaneous administration of 15 micrograms of Vaxigrip im and 10⁵ IU of IFN-α ip.

### DETAILED DESCRIPTION OF THE INVENTION

The present invention is directed to a method for downregulating pathogenic immune reactions by locally administering interferon at a site remote from that of the immune reaction. The interferon is administered in an amount that is effective to induce trafficking of antigen presenting cells to the site of interferon administration.

The immune reaction can be an ongoing immune reaction, or it can be an incipient or prospective immune reaction. An ongoing immune reaction may be an allergic attack or it may be anaphylactic shock that results from exposure to an antigen to which the patient is hypersensitive, or it may be nearly any incidence of undesirable inflammation. An attack of atopic asthma or allergic rhinitis are further examples of an ongoing immune reaction that may be downregulated according to the present invention.

Among the ongoing immune reactions that may be treated in accordance with the present invention are autoimmune diseases. Autoimmune diseases, by definition, involve an immune response against a self-antigen or antigens. Autoimmune diseases also often have an inflammatory component, for example, pancreatitis and insulinitis in Type I diabetes. Pancreatitis and insulinitis are inflammatory diseases of the pancreas, often observed in patients with incipient diabetes prior to the clinical manifestation of diabetes, and may also be treated in accordance with the present invention.

The present invention is particularly appropriate for the treatment of any chronic inflammatory disease. Crohn's disease is a result of an abnormal immune response of the gut mucosa triggered by one or more environmental risk factors in people with predisposing gene variations. Thus, downregulation of such immune response would be expected to be beneficial to a patient with Crohn's disease or patients with other forms of inflammatory bowel disease.

Another indication for treatment in accordance with the present invention is the abnormal reaction to vaccinations such as smallpox or Bacillus Calmette-Guérin (BCG) vaccination. This reaction manifests itself in severe inflammation in the area of the vaccination site, which can spread uncontrollably and even result in death. This is a type of immune reaction that can be treated by means of the present invention.

To downregulate the immune response in accordance with the present invention, the interferon is locally administered at a site remote from the site of administration of the protein in question. In the case of an ongoing pathogenic immune reaction, the interferon can be administered at any time during the course of that reaction in order to downregulate it.

With respect to an incipient or prospective immune reaction, this relates to the aspect of the present invention by which the immunogenicity of an expected immune reaction to the administration of, for example, a therapeutic compound or composition can be diminished. For example, it is known that administration of a non-human antibody for therapeutic purposes often results in an immune reaction against that antibody, sometimes referred to as a HAMA, or Human Anti-Mouse Antibody, reaction. Even when a humanized antibody is administered, there is often an immune reaction to those portions of the antibody that are not human, and, indeed, therapeutic administration of a totally human antibody can often lead to an anti-idiotypic immune reaction. The administration of any therapeutic protein or polypeptide can elicit an immunogenic response that might either neutralize the activity of the administered protein, or at least adversely alter its pharmacokinetics, thus drastically affecting the serum half-life of the protein, resulting in the necessity of increased dose or more frequent dosing, both of which lead to increased expense and the risk of adverse events and reduced patient compliance.

In the case of the reduction of the severity of the immune reaction to the administration of an immunogenic therapeutic, the administration of interferon should be concurrent with the administration of the therapeutic that can provoke the immune reaction. The term “concurrent” means that the two administrations may be substantially simultaneous, or the interferon may be administered plus or minus 2-3 hours from the administration of the protein. The longer the separation is in time, then the less effective is the downregulation caused by the interferon. Preferably, the interferon is administered within half an hour, more preferably within 15 minutes, and most preferably within 5 minutes of administration of the protein. Thus, the term “concurrently” must be understood within these time parameters. Within such parameters, if the protein is administered first, then the reaction is an incipient reaction, and if the interferon is administered first, then the reaction is a prospective reaction.

Other conditions in need of downregulation of pathogenic immune reactions include organ or tissue transplantation. With regard to organ or tissue transplantation, the method may be considered to be reducing the severity of an incipient or prospective immune reaction, or it could be considered to be downregulation of an ongoing pathogenic immune reaction, depending upon the time that the interferon is administered. The interferon may be administered concurrently with the transplant, as this term is defined in the previous paragraph. The interferon can also be administered any time after the transplantation. If no signs of rejection or other abnormal immune response to the transplant has yet manifested itself, then the administration will be for the purpose of reducing the severity of a prospective immune reaction. Such administration may be administered for an indefinite period of time. Once rejection or other abnormal immune reaction to the transplant has manifested itself, then the administration of the interferon would be for the purpose of downregulating an ongoing pathogenic immune reaction, and again such administration can be continued as long as is necessary.

The interferon must be administered at a site other than the site of the ongoing, incipient or prospective immune reaction. Thus, if the reaction in question results from the intramuscular or subcutaneous administration of a protein, for example, then the interferon should be administered at a site that is remote from the site of administration of the protein. Thus, for example, if the protein is administered in the right arm, the interferon can be administered in the left arm or in a posterior muscle. If the ongoing pathogenic immune reaction is in a particular type of tissue or organ, the interferon should be administered intramuscularly in a suitable location that is remote from the organ or tissue that is affected. By “remote”, it is not necessarily meant that the site of IFN administration must be at a specific physical distance from the site of the immune reaction. It should be remote in the sense that the immune components must travel a long way to get from one to the other, or a different pathway must be taken to get from the source of such immune components to the site of the immune reaction, on the one hand, and the site of interferon administration, on the other.

Without being limited, it is understood that at least part of the mechanism of action of the present invention involves the trafficking of antigen presenting cells to the site of interferon administration, and away from the site of the immune reaction. This is why it is important that the site of interferon administration should be as far as reasonably possible from the site of immune reaction. Thus, the term “remote from”, as used in the present specification and claims, means not so close as to prevent the downregulation that is the object of the present invention.

The amount of interferon to be administered is an amount to induce trafficking of antigen presenting cells to the site of interferon administration. As indicated above, it is also expected that this trafficking will be away from the site of the immune reaction. It is expected that there will be an optimum amount of interferon to provide the optimum results. Thus, the interferon may be administered in an amount from about 10⁵ to about 5×10⁷ IU, more preferably between about 10⁶ and about 2×10⁷ IU, and most preferably between about 5×10⁶ and about 10⁷ IU.

The interferon is preferably parenterally administered, and more preferably administered intramuscularly, subcutaneously, transdermally or intraperitoneally. Because it is important that the administration be at a defined location, i.e., local, one should not administer the interferon in a manner such that it is rapidly dispersed within the body, such as orally, intravenously, oromucosally, mucosally, or per os. The interferon must be administered so as to have a local effect.

Thus, for the purpose of the present specification and claims, “local” administration, or administration “locally”, means that the interferon is administered in such a manner that it will remain in one location long enough to permit the trafficking of antigen presenting cells to the site of interferon administration. While intramuscular administration, for example, is generally thought of as a means of systemic administration, for the purpose of the present invention it is a means of local administration as the interferon is not rapidly dispersed from the site of the intramuscular administration.

Another mode of administration of the interferon is by means of a patch that has some type of transdermal administration modality, such as microneedles.

As the interferon being administered in accordance with the present invention is not being administered for its direct antiviral or antineoplastic effect, but predominantly for its effect in causing trafficking of antigen presenting cells, it is believed that this effect may be achieved by the administration of any Type I interferon. Type I interferons are encoded by genes devoid of introns, and include the IFN-α family, of at least 13 functional IFN-α subtypes, IFN-β and IFN-ω. These interferons are produced effectively by all cell types.

The following are non-limiting examples of sources of IFN that can be used in the present invention: natural IFN-α (a mixture of different IFN-α subtypes or individual IFN-α subtypes), from stimulated leukocytes of healthy donors, or lymphoblastoid IFN-α from Namalwa cells; a synthetic type I IFN, such as consensus IFN (cIFN); recombinant IFN-β (commercially available as Rebif™, Serono; Avonex™, Biogen; and β-Seron™, Berlex), or recombinant IFN-α subtypes, such as IFN-α2a (commercially available as Roferon™, Roche), and IFN-α2b (commercially available as Intron-A™, Schering Plough), or IFN-ω; new IFN molecules generated by the DNA shuffling method, or site-directed mutagenesis, provided that they are used in the above-mentioned dosages indicated per vaccine dose; and recombinant human IFN-α or IFN-β molecules having one or more amino acid substitutions, deletions or additions, or otherwise obtained with naturally occurring polymorphisms such as the polypeptides of GenOdyssee, WO 02/101048 (see also WO 02/083733, WO 03/000896, and others).

The interferon may be administered in the form of a pharmaceutical composition with standard excipients. In the pharmaceutical composition administered, the interferon dosage form of the present invention may include a variety of vehicles and excipients for IFN, as will be apparent to the skilled artisan. Representative formulation technology is taught in, inter alia, Remington's Pharmaceutical Sciences (1995), and its predecessor and successor editions. The IFN formulation may comprise stability enhancers, such as glycine or alanine, as described in U.S. Pat. No. 4,496,537, and/or one or more carriers, such as a carrier protein. For example, for treatment of humans, pharmaceutical grade serum albumin, optionally together with phosphate-buffered saline as diluent, is commonly used. Where the excipient for IFN is human serum albumin, the human serum albumin may be derived from human serum, or may be of recombinant origin. Normally, when serum albumin is used it will be of homologous origin.

It should be understood that the present invention is not related to the phenomenon of oral tolerance. Thus, for example, it has been reported in Nelson et al (1996), that ip administration of IFN-β enhances the suppressive effect of oral myelin antigens when administered to suppress EAE (Experimental Autoimmune Encephalitis). The oral myelin antigens are not being administered for the purpose of provoking an immune reaction in Nelson. The interferon is being administered to enhance the suppressive effects of the orally administered antigen, not to directly reduce any immune reaction to the antigen. Thus, the interferon is always administered in conjunction with the oral administration of antigen intended to induce tolerance. The present invention specifically excludes the concurrent administration of the interferon with an orally administered antigen for the purpose of inducing tolerance.

Although the humoral and cellular immune systems play an important role in limiting the spread of infections and the development of neoplastic diseases, an excessive or abnormal immune reaction can often be pathogenic and detrimental to the health of the patient. The present invention is not directed to the downregulation of beneficial immune reactions, but only to the downregulation of pathogenic immune reactions, i.e., excessive or abnormal immune reactions. Accordingly, the administration of interferon for the known indications of the treatment of viral infections or neoplastic conditions would not be examples of methods for downregulating an ongoing pathogenic immune response, nor would such patients be in need of having an ongoing pathogenic immune response downregulated. Indeed, one would not be motivated to treat a viral or neoplastic disease by downregulating the immune response thereto, because it is often the very immune response that provides the best protection against the viral or neoplastic disease. Thus, there would be no motivation to treat a viral infection by means of the present invention except in the case of a viral infection that involves a pathogenic immune response. An example of the latter type of virus is lymphocytic choriomeningitis virus. The pathology of this virus resides in the overreaction of the immune response thereto. It would not have been expected from the known general antiviral activity of interferon, that the treatment of this virus by local administration of interferon would have a significant effect in treating the pathology by downregulating the immune response. Thus, the treatment of this virus is included in the present invention as it is the pathogenic immune reaction that is being treated and such would not be obvious from prior art generally disclosing the antiviral effects of Type I interferon.

While IFN-α has been approved for the treatment of hepatitis C, the inclusion/exclusion criteria therefor provides that the IFN is only administered prior to end-stage liver disease characterized by fibrosis and non-compensated cirrhosis. Thus, the IFN is only administered prior to the time that the immune reaction to the hepatitis C virus becomes pathogenic. In the present invention, the administration of IFN is only after a pathogenic immune response asserts itself as it is only then that the patient is in need of downregulation of an ongoing pathogenic immune response. In one embodiment of the present invention, the treatment of hepatitis C is excluded. In another embodiment of the present invention, the treatment of immune reactions to viral infections in general is excluded.

Type I interferon has been approved for the treatment of certain types of multiple sclerosis. It is thought that the primary mechanism of action for this indication is the downregulation of both the production and activity of IFN-y in the brain. While this is not the same type of immune system downregulation that is the basis of the present invention, nevertheless, in accordance with one embodiment of the present invention, the treatment of multiple sclerosis is disclaimed as being part of the present invention. Thus, for the purpose of one embodiment of the present invention, the patient to whom the interferon is administered is other than one with multiple sclerosis.

As indicated above, the effect of the interferon for the purpose of the present invention is dependent upon the localization thereof at a particular location. It is not necessary for the desired effect in accordance with the present invention for the interferon to eventually enter the bloodstream. As interferon has other effects when systemically administered, it may be desirable to administer the interferon in such a way as to prevent or delay entry into the bloodstream. Thus, the present invention further comprehends the implantation of an insoluble substrate, to which interferon is conjugated in such a way as to maintain its biological activity, but prevent its solubility. This would also have the beneficial effect of increasing its residence time at the local site, which is remote from the site of the ongoing immune response. See, for example, U.S. Pat. No. 4,041,152 to Chany et al, which discloses how to make biologically active interferon that is immobilized on a solid support.

Any form of interferon that would serve to delay the systemic dispersion, i.e., increase the local residence time, would be particularly useful for the purposes of the present invention. Interferon coupled to an insoluble or soluble high molecular weight support is known and very advantageously used in the present invention. An example is Albumin-IFN-α (Albuferon-Alpha™, Human Genome Sciences); see www.hgsi.com/products/albuferon.html and U.S. patent publication US 2003/0199043. Other examples are the sustained release interferon formulations of U.S. Pat. Nos. 4,774,091 and 4,855,134.

The effect of the present invention may be potentiated by administering the interferon at a plurality of sites, each of which are remote from the site of the immune response. Such a multiple mode of administration would be expected to enhance the trafficking of the antigen presenting cells away from the immune reaction. When treating a chronic condition or when the interferon must otherwise be administered over a matter of days or weeks, the site of administration may be varied over time so as to avoid possible side effects.

### EXAMPLES

### Example 1

### Adjuvant Activity of Interferon Alpha: Mechanism(s) of Action

### Materials and Methods

Recombinant mouse IFN-α was purchased from PBL Laboratories, Piscataway N.J., USA. ISRE-EGFP transgenic mice containing an enhanced green fluorescent protein (EGFP) reporter gene under the control of an interferon responsive chimeric promoter, on a C57Bl/6 background, were maintained as a specific pathogen-free breeding colony at the Institut Andre Lwoff Villejuif. Specific pathogen-free C57Bl/6 mice were purchased from Iffa Credo, L'Arbresles, France. Influenza-specific total IgG, IgG1, IgG2a and IgA levels were determined by ELISA as described previously (Proietti et al, 2002) except that the plates were coated with 1.0 g/ml of Vaxigrip™, Avantis Pasteur MSD, Lyon, France.

### Results and Discussion

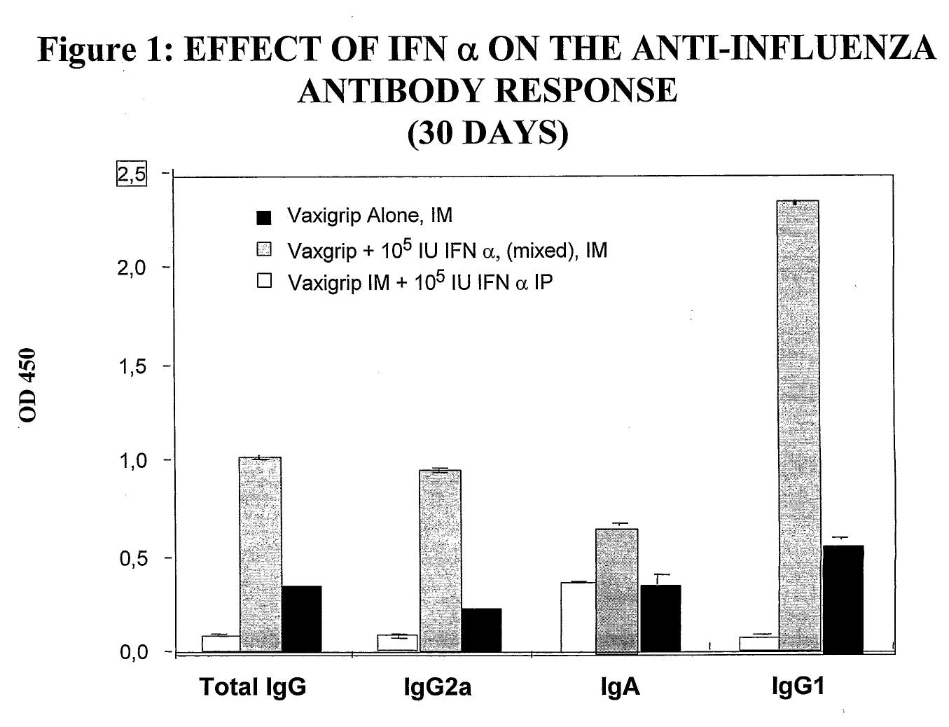

Treatment of C57Bl/6 mice with recombinant IFN-α was found to markedly enhance the humoral response to a commercially available influenza vaccine (Vaxigrip™) when ad-mixed with the vaccine and injected intramuscularly (FIG. 1; compare center and right columns of each graph). In agreement with a previous report (Jego et al, 2003), IFN-α treatment was found to markedly increase the serum levels of all four classes of influenza-specific immunoglobulins (IgG, IgG1, IgG2a, and IgA) tested (FIG. 1; compare center and right columns of each graph). The effect of IFN-α on influenza-specific Ig production was dose-dependent with the greatest increase in Ig sub-classes observed at a dose of 10⁵ IU, the highest dose of IFN tested. In contrast, ip injection of the same dose of IFN-α that is distant to the site of vaccination (hind leg) was found to markedly inhibit the antibody response to influenza vaccination (FIG. 1; note left column of each graph). Indeed, the levels of all the sub-classes (IgG, IgG1, IgG2a, and IgA) of influenza-specific Ig tested following ip injection of IFN-α in animals vaccinated im with Vaxigrip™ were markedly lower than those observed in animals vaccinated im with Vaxigrip™ alone (FIG. 1: compare left and right columns of each graph).

The use of transgenic mice expressing an enhanced green fluorescent protein (EGFP) reporter gene regulated by an IFN responsive chimeric promoter has shown that IFN activated cells are present in the peripheral circulation of influenza vaccinated mice as early as 4 hours after initiation of IFN-α treatment. The principal cell populations activated by IFN treatment included myeloid dendritic cells (B220(−), Ly6c(−), CD11c(high), CD11b(high)) and plasmacytoid dendritic cells (B220(high), Ly6c(high), CD11c(low), CD11b(−)) (data not shown).

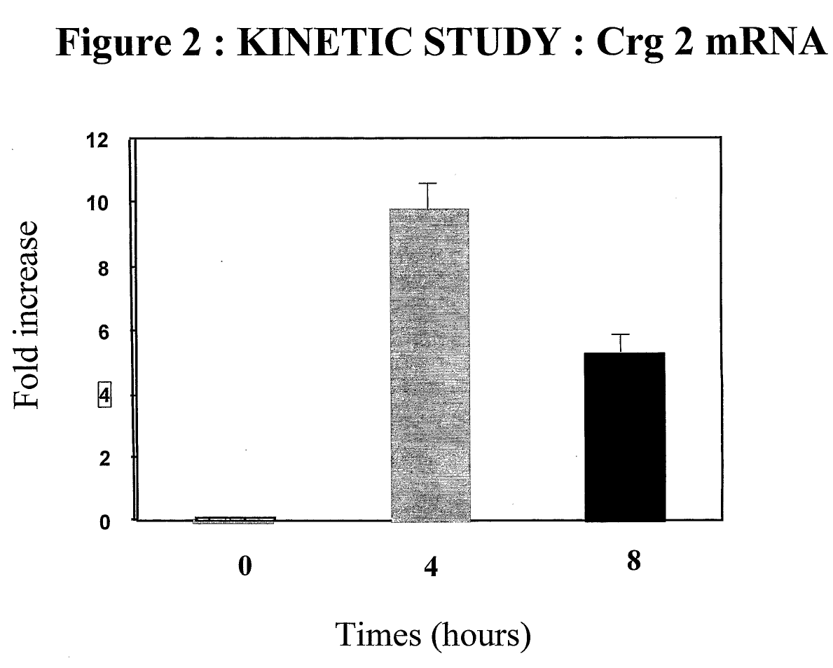

Ip injection of IFN-U at a site distant to the site of influenza vaccination was found to markedly increase the number of IFN activated cells present in the peritoneum, suggesting that the marked inhibition of the influenza-specific Ig response to influenza vaccination observed under these conditions may be related to trafficking of IFN-activated antigen-presenting cells away from the site of influenza vaccination. Differential display analysis showed that numerous IFN responsive genes were induced in the lymphoid tissue of IFN treated animals including Crg2 (FIG. 2) and other chemokines which regulate lymphocyte trafficking, proteases associated with antigen processing, and genes involved in lymphocyte activation, apoptosis, and protein degradation (data not shown). Thus, together these results suggest that the mechanisms underlying the immuno-suppressive activity of IFN-α involve trafficking of antigen presenting cells towards the site of vaccination.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Balance et al, “Albumin Fusion Proteins”, US 2003/0199043 (Oct. 23, 2003)
Chany et al, “Pharmaceutical Formulation of Interferon Insolubilized by Fixation on a Support”, U.S. Pat. No. 4,041,152 (Aug. 9, 1997)
Diebold et al, “Innate antiviral responses by means of TLR7-mediated recognition of single-stranded RNA”, Science 303(5663):1529-1531 (2004)
Escary J-L, “New Polynucleotides and Polypeptides of the IFNU-6 Gene”, WO 02/083733 (Oct. 24, 2002)
Escary J-L, “New Polynucleotides and Polypeptides of the IFNU-7 Gene”, WO 02/101048 (Dec. 19, 2002)
Escary J-L, “New Polynucleotides and Polypeptides of the IFN$G(a)-5 Gene”, WO 03/000896 (Jan. 3, 2003)
Gennaro A R (ed.), Remington's Pharmaceutical Sciences, l₉th Ed. Mack Publishing Company (Easton, Pa., 1995)
Heil et al, “Species-specific recognition of single-stranded RNA via toll-like receptor 7 and 8”, Science 303(5663):1526-1529 (2004)
Jego et al, “Plasmacytoid dendritic cells induce plasma cell differentiation through type I interferon and interleukin 6”, Immunity 19(2):225-234 (2003)
Kwan HK, “Biologically Stable Alpha-Interferon Formulations”, U.S. Pat. No. 4,496,537 (Jan. 29, 1985)
Le Bon et al, “Type i interferons potently enhance humoral immunity and can promote isotype switching by stimulating dendritic cells in vivo”, Immunity 14(4):461-470 (2001)
Mayer et al, “Therapeutic potential of oral tolerance”, Nat Rev Immunol 4(6):407-419 (2004)
Medzhitov R, “CpG DNA: security code for host defense”, Nat Immunol 2(1):15-16 (2001)
Nelson et al, “Effect of oral beta interferon on subsequent immune responsiveness”, Ann N Y Acad Sci 778:145-155
Proietti et al, “Type I IFN as a natural adjuvant for a protective immune response: lessons from the influenza vaccine model”, J Immunol 169(1):375-83 (2002)
Yamahira et al, “Long-Term Sustained-Release Preparation”, USP (Sep. 27, 1988)
Yamahira et al, “Sustained-Release Preparation, U.S. Pat. No. 4,855,134 (Aug. 8, 1989)

What is claimed is:

1. A method for downregulating an ongoing pathogenic immune reaction or reducing the severity of an incipient or prospective immune reaction in a patient in need thereof, comprising:
in the course of the ongoing immune reaction, or concurrently with the provocation of an immune reaction, locally administering, at a site remote from the site of the ongoing, incipient or prospective immune reaction, an amount of Type I interferon effective to induce trafficking of antigen-presenting cells to the site of interferon administration, with the proviso that the patient is not one with multiple sclerosis, and with the further proviso that the interferon is not concurrently administered with the oral administration of an antigen for the purpose of inducing tolerance.

2. A method in accordance with claim 1, where the patient in need is one in need of downregulation of an ongoing immune reaction, and said administering step is accomplished in the course of the ongoing immune reaction.

3. A method in accordance with claim 1, where the patient in need is one in need of reduction of the severity of an incipient or prospective immune reaction, and said administering step is accomplished concurrently with the provocation of an immune reaction.

4. A method in accordance with claim 2, wherein said patient in need is one suffering from an allergic attack, atopic asthma, allergic rhinitis, or anaphylactic shock that results from exposure to an antigen to which the patient is hypersensitive.

5. A method in accordance with claim 2, wherein said patient is one suffering from an undesirable inflammation.

6. A method in accordance with claim 5, wherein said patient is one suffering from a chronic inflammatory disease.

7. A method in accordance with claim 6, wherein said patient is one suffering from an inflammatory bowel disease.

8. A method in accordance with claim 2, wherein said patient is one suffering from an autoimmune disease other than multiple sclerosis.

9. A method in accordance with claim 3, wherein the incipient or prospective immune reaction is that which is in response to the administration of a therapeutic protein that is immunogenic.

10. A method in accordance with claim 1, wherein said interferon is administered in an amount from about 10⁵ to about 5×10⁷ IU.

11. A method in accordance with claim 1, wherein the interferon is administered intramuscularly, subcutaneously, transdermally, or intraperitoneally.

12. A method in accordance with claim 1, wherein the Type I interferon is interferon-α.

13. A method in accordance with claim 1, wherein the Type I interferon is interferon-β.

14. A method in accordance with claim 1, wherein the interferon is conjugated to an insoluble substrate in such a way as to maintain its biological activity, but prevent its solubility.

15. A method in accordance with claim 1, wherein the patient in need is other than one suffering from hepatitis C.

16. A method in accordance with claim 1, wherein the patient in need is other than one suffering from a viral infection.
